(19) United States
(12) Patent Application Publication
(10) Pub. No.: US 2011/0135630 A1
(43) Pub. Date: Jun. 9, 2011

(54) CHEMICAL MODIFICATION OF ANTIBODY FRAGMENTS

(76) Inventor: Thomas J. Tolbert, Bloomington, IN (US)

(21) Appl. No.: 12/834,758

(22) Filed: Jul. 12, 2010

Related U.S. Application Data
(60) Provisional application No. 61/225,208, filed on Jul. 13, 2009.

(51) Int. Cl. A61K 39/395; C12P 21/04; C07K 16/00; C12N 5/00; A61P 35/00

(52) U.S. Cl. 424/130.1; 435/69.6; 530/387.1; 435/375

(57) ABSTRACT

The present invention provides methods for expressing glycoproteins comprising a N-terminal cysteine. Methods are also provided for chemically modifying the glycoproteins comprising a N-terminal cysteine.

### BACKGROUND OF THE INVENTION

The present invention generally relates to methods for chemically modifying glycoprotein fragments and more specifically, to methods for chemically modifying glycoprotein fragments by native chemical ligation.

Site-selective chemical modification of proteins has wide application in biochemistry and biotechnology including the incorporation of labels and defined post-translational modifications onto proteins to facilitate biochemical studies, and incorporation of synthetic modifications onto proteins to alter bioactivity and physical properties.

Native chemical ligation (NCL) is a chemical reaction between N-terminal cysteines and thioesters which forms native peptide bonds (Dawson, P E et al., Science 1994, 266, 776-779). The N-terminal cysteines may be native to the protein fragment of interest or they may be engineered into the amino acid sequence. One common way of engineering a protein fragment with an N-terminal cycteine is overexpression of the desired protein fragment. However, methods for expression of glycoproteins containing N-terminal cysteines have not been reported.

Therefore it would be desirable to have a method for expression of glycoproteins containing N-terminal cysteines that may then be modified by native chemical ligation.

### SUMMARY OF THE INVENTION

In one aspect of the present invention there is provided a method for expression of a glycoprotein comprising a N-terminal cysteine, the method comprising the steps of inserting a nucleotide sequence encoding for the glycoprotein into a plasmid to form a plasmid construct, wherein the nucleotide sequence comprises a codon of cysteine at the N-terminus and wherein the plasmid construct further comprises nucleotide sequences encoding a N-terminal signal peptide and an α-factor, transforming the plasmid construct into a glycosylation deficient strain of yeast and culturing the yeast transformed with the plasmid construct to express the glycoprotein.

In another aspect of the present invention there is provided a method of making a chemically modified glycoprotein fragment comprising the steps of providing a glycoprotein comprising a N-terminal cysteine and reacting the glycoprotein with a ligand, wherein the ligand comprises a thioester.

These and other features, aspects and advantages of the present invention will become better understood with reference to the following drawings, description and claims.

### BRIEF DESCRIPTION OF THE DRAWINGS

FIG. 1 is a schematic showing C-IgG1 Fc 3 protein expression;

FIG. 2 is a SDS-PAGE gel of C-IgG1 Fc 3;

FIG. 3 is the ESI Mass Spec of heterogeneously glycosylated C-IgG1 Fc 3;

FIG. 4A is the ESI Mass Spec of deglycosylated hydroxylamine treated C-IgG1 Fc;

FIG. 4B is the ESI Mass Spec of deglycosylated C-IgG1 Fc;

FIG. 5 is the ESI Mass Spec of C-IgG1 Fc 4;

FIG. 6 is a synthesis scheme for cyclic-RGDfK 1 and cyclic-RGDfK-thioester 2;

FIG. 7A is an analytical HPLC trace for cyclic-RGDfK-thioester 2;

FIG. 7B is an HRMS ESI-TOF of cyclic-RGDfK-thioester 2;

FIG. 8 is the ESI Mass Spec of C-IgG1 Fc 5;

FIG. 9 is cyclic-RGDfK α_{V}β₃integrin antagonist 1;

FIG. 10 is a scheme showing the solid phase synthesis of cyclic-RGDfK-thioester 2; and

FIG. 11 is a schematic showing the Conversion of heterogeneously glycosylated C-IgG1 Fc (3) to RGD-Man₅-IgG1 Fc (5) by mannosidase IA digestion and NCL with thioester 2.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides methods for expressing a glycoprotein containing a N-terminal cysteine. The glycoprotein may be a glycoprotein fragment. The glycoprotein having an N-terminal cysteine may then be used to produce a chemically modified glycoprotein through nativel chemical ligation (NCL). The glycoprotein may be reacted with a ligand having a thioester, resulting in an amide linkage between the glycoprotein and the ligand. The type of the glycoslylation of the glycoprotein may then be altered by treatment with enzymes to produce different glycosylated forms. The methods of the present invention are applicable to glycoproteins such as antibody fragments that have significant N-glycosylation.

In one embodiment of the present invention, a method for expression of a glycoprotein containing an N-terminal cysteine is provided. The method has the steps of inserting a nucleotide sequence encoding for the glycoprotein into a plasmid to form a plasmid construct, where the nucleotide sequence has a codon for a N-terminal cysteine. The codon for the N-terminal cysteine may be part of the native nucleotide sequence or it may be inserted by either mutating a codon for another amino acid to that of cysteine or adding a codon for cysteine at the N-terminus. The codons for cysteine are TGT or TGC. The nucleotide sequence may encode for an entire glycoprotein or for a fragment of a glycoprotein. In an exemplary embodiment the nucleotide sequence may encode for a glycoprotein fragment such as the Fc domain of IgG.

In another embodiment, the plasmid construct may further comprise nucleotide sequences encoding for an N-terminal signal peptide and a secretion factor, such as but not limited to, an α-factor, where these nucleotide sequences are fused to the nucleotide sequence encoding the glycoprotein. The N-terminal signal peptide directs the glycoprotein into the secretory pathway and the α-factor is a secretion signal, allowing for secretion of the glycoprotein. Plasmids are known in the art incorporating nucleotide sequences for the N-terminal signal peptide and α-factor. The nucleotide sequence of the glycoprotein of interest may be inserted into the plasmid to provide a fusion protein of N-terminal signal peptide-α-factor-glycoprotein. A non-limiting example may be plasmid pGAPzαA (Invitrogen).

In a further embodiment, the plasmid construct is transformed into a glycosylation deficient strain of yeast by methods commonly known in the art. By glycosylation deficient it is meant that the glycoprotein will still be glycosylated by oligosaccharide transferase (OST) with the addition of Man₈₋₁₂GlcNac₂ after translation of the nucleotide sequence. The glycosylation deficient strain of yeast may be an OCH1 deleted strain of yeast where the och1 gene has been deleted from the yeast genome. Och1 encodes for α-1,6-mannosyltransferease, the enzyme responsible for providing hypermannosylated N-glycan structures with greater than 100 mannose residues. By deleting OCH1 the glycoprotein expressed with the methods of the present invention may be limited to from between about 8-12 mannose residues. The glycosylation deficient strain of yeast may be obtained by the skilled artisan without undue experimentation using methods described in the literature. Examples of articles describing methods for making glycosylation deficient strains of yeast are Nett, J H & Gerngross, T. U, Yeast 2003, 20, 1279-1290 and Choi, B-K. et al., Proc. Natl. Acad. Sci. USA, 2003, 100, 5022-5027, incorporated by reference herein. The glycosylation deficient strain of yeast may be a strain of Saccharomyces cerevisiae or Pichia pastoris where the och1 gene has been knocked out.

After transformation of the glycosylation deficient yeast strain, cells expressing the glycoprotein containing the N-terminal cysteine are selected by methods known in the art. Once the cells are selected they may then be cultured under conditions for the selected yeast strain. The glycoprotein will be secreted and can be isolated from the culture media. The steps for purifying the glycoprotein will be determined based on the glycoprotein expressed. The N-terminal cysteine does not affect the purification so any purification scheme known for the desired glycoprotein may be used.

In one embodiment methods are provided for chemically modifying the glycoprotein having a N-terminal cysteine. The glycoprotein may be modified using native chemical ligation (NCL). The glycoprotein is reacted with a ligand where the ligand has a thioester. The thioester reacts with the sulfydryl of the cysteine and after rearrangement the ligand is covalently attached to the glycoprotein through an amide bond. The ligand may be any molecule. It may be a peptide, a vitamin, a receptor agonist or antagonist, a substrate, an inhibitor or a drug. It may be derivatized with a thioester such that the molecule is not prohibited from serving the desired function once it is attached to the glycoprotein.

In a further embodiment, there are provided methods to alter the glycosylation of the chemically modified glycoprotein by treatment with enzymes to produce different homogeneous glycosylated forms. Certain glycosylated forms of the Fc region of IgG have been shown to have increased antibody-dependent cell-mediated cytotoxicity and complement dependent cytotoxicity. Therefore it would be desirable to alter the glycosylation of the Fc region to increase these cytotoxic immune responses. The glycosylation of chemically modified Fc regions of IgG may be altered by treatment with enzymes such as glycosidases and glycosyltransferases that modify the oligosaccharide structure of the chemically modified glycoprotein.

In an exemplary embodiment the glycoprotein is an Fc region fragment of IgG. The Fc region is known effect antibody dependent cell-mediated cytotoxicity and complement dependent cytotoxicity. Therefore it would be desirable to have a way to selectively bind the Fc region to undesired cells such as cancer cells. Receptors, such as α_{V}β₃-integrin receptor and folate receptors are known to be overexpressed on cancer cells. The Fc region of IgG having an N-terminal cysteine may be covalently modified with a cyclic RGD peptide, a folate molecule or a folate mimetic, where each have been derivatized to have a thioester moiety.

In yet another embodiment, there are provided methods for using the chemically modified glycoprotein of the present invention. The chemically modified glycoprotein may be used to destroy unwanted cells such as cancer cells by contacting the cells with a effective concentration of a chemically modified glycoprotein. An effective amount of chemically modified glycoprotein may be easily determined by the skilled artisan. For example, an effective amount may be an amount that results in an IC₅₀ of greater than about 20%, about 40%, about 50%, about 60%, about 70%, about 80% or about 90%.

Methods for treating a patient having cancer comprise administering to the patient a therapeutically effective amount of a chemically modified glycoprotein fragment. A therapeutically effective amount may be determined based on the type of cancer and the severity. A therapeutically effective amount may be determined by the physician and may be adjusted during the course of therapy as needed. The chemically modified glycoprotein may combined with another drug or therapeutic. It may also be administered in a pharmaceutically acceptable carrier and administered in an appropriate manner.

### EXAMPLES

### Materials and Methods

Materials: Chemicals were obtained from Sigma-Aldrich unless otherwise noted. For peptide synthesis, Fmoc amino acids were obtained from EMD-Novabiochem and AAPPTEC, DEPBT was obtained from AAPPTEC, and TFA was obtained from J T Baker. High resolution ESI mass spectrometry was performed on a Waters/Micromass LCT Classic. Low resolution ESI mass spectrometry was performed on a PE Sciex API III. Mouse mannosidase IA was expressed in Pichia pastoris and purified as described by Tempel at al. (J. Biol. Chem. 2004, 279, 29774-29786). PNGase F was expressed and purified in E. coli as described by Loo et al. (Protein Expression Purif. 2002, 24, 90-98). Human melanoma WM-115 cells were obtained from the American Type Culture Collection (ATCC, Manassas, Va.). WM-115 cells were cultured in Eagle's minimum essential medium (E-MEM) supplemented with 10% fetal bovine serum (FBS), 1% nonessential amino acids, 1% sodium pyruvate, 100 U/ml penicillin, and 100 U/ml streptomycin at 37° C. in a humidified atmosphere containing 5% CO₂. E-MEM, FBS, nonessential amino acids, sodium pyruvate, penicillin, and streptomycin were obtained from Hyclone Laboratories, Inc.

### Production and Characterization of C-IgG1 Fc 3 and C-Man₅-IgG1 Fc 4:

Cloning of C-IgG1 Fc. DNA encoding the Fc region of human immunoglobulin heavy chain constant gamma 1 was amplified by PCR using plasmid MGC-12853 from the mammalian gene collection (Strausberg, R. L. et al., Science 1999, 286, 455-457) as a template and the following PCR primers:

5′-XhoI-CTC-IgG Fc (5′-ggcccgctcgagaaaagatgcacatgcccaccgtgcccagca-3′) (SEQ ID 1)

3′-NotI-stop-IgG Fc (5′-gggcccgcggcggccgctcatttacccggagacagggagag-3′) (SEQ ID 2)

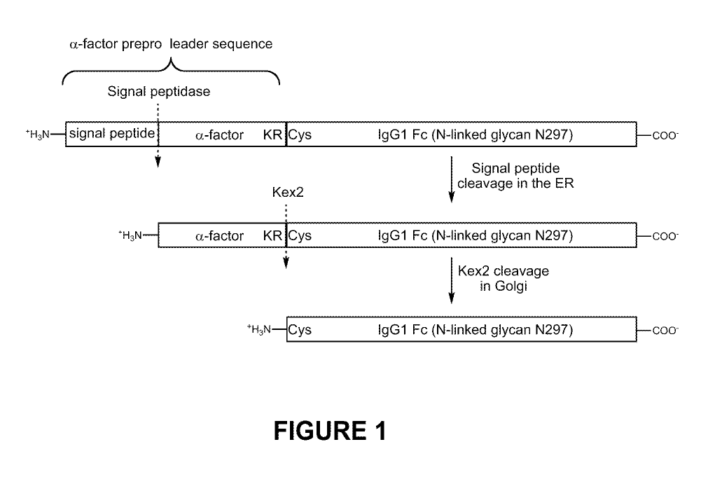

The resulting PCR product and plasmid pGAPzαA (Invitrogen) were digested with Xho I and Not I restriction endonucleases, and then ligated together using T4 DNA ligase. The ligated DNA was transformed into Top10F′ E. coli (Invitrogen) by electroporation and selected on low salt LB/zeocin plates (1% peptone, 0.5% yeast extract, 0.5% NaCl, 1.5% agar, +25 μg/mL Zeocin). Plasmid pGAP-CTC-IgG Fc was isolated from a positive clone and insertion of the correct C-IgG1 Fc encoding DNA was confirmed by sequencing. The pGAP-CTC-IgG Fc plasmid encodes for the C-IgG1 Fc protein fused to the α-factor prepro leader sequence from pGAPzαA to direct protein secretion (FIG. 1).

Transformation of pGAP-CTC-IgG Fc into a glycosylation deficient strain of SMD1168 Pichia pastoris. Plasmid pGAP-CTC-IgG Fc was linearized by restriction digestion with Avr II and transformed by electroporation (Wu, S. et al., BioTechniques 2004, 36, 152-154) into an OCH1 deleted strain of SMD1168 Pichia pastoris produced previously in our laboratory using the methods of Nett and Gerngross (Yeast 2003, 20, 1279-1290). Colonies were selected on YPD/Zeocin plates (1% yeast extract, 2% peptone, and 2% glucose, 2% agar, +100 μg/mL Zeocin) and positive colonies containing human IgG1 Fc DNA were confirmed by PCR.

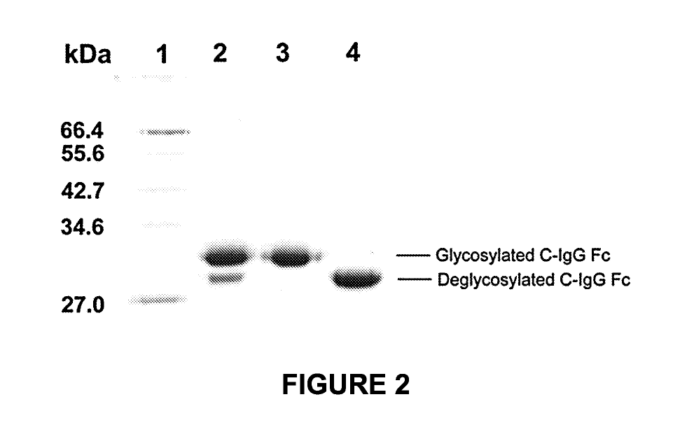

Expression and purification of heterogeneously glycosylated C-IgG1 Fc 3. C-IgG1 Fc was expressed utilizing the glycosylation deficient, constitutive IgG1 Fc expressing strain of SMD1168 Pichia pastoris produced as described above. A 2 mL YPD/Zeocin culture (YPD: 1% yeast extract, 2% peptone, and 2% glucose, +100 μg/mL Zeocin) was grown at 25° C. for 72 h, and then inoculated into a 50 mL YPD/Zeocin culture and grown at 25° C. for another 72 h. The dense 50 mL YPD/Zeocin culture was then inoculated into a spinner flask containing 1 L of supplemented YPD media (YPD+4×10⁻⁵% biotin+0.004% histidine). During yeast cell growth the media was aerated by addition of air through a sterilized glass sparger through a sterile 0.2 micron filter at a rate of 1 L/min. The 1 L culture was grown at 25° C. for 24 h to allow the culture to grow to density. Thereafter, 50 mL of a sterile 20% glucose solution was added into the 1 L culture every 24 h for 3 days. The culture supernatant was then separated from yeast cells by centrifugation at 5400 g for 10 min at 4° C. The pH of the supernatant was adjusted to 7 and the resulting solution was incubated at 4° C. for 2 h and then filtered. The filtered supernatant was loaded onto a protein G column (5 mL bed volume, pre-equilibrated with 20 mM sodium phosphate pH 7.0). The column was washed with 50 mL of 20 mM sodium phosphate pH 7.0. The C-IgG1 Fc 3 was then eluted with a 0.1 M glycine solution at pH 2.7 and dialyzed against 20 mM sodium phosphate buffer pH 7.0 at 4° C. overnight. The amount of C-IgG1 Fc 3 obtained from protein G purification of 1 L of culture was about 30 mg as determined by absorbance at 280 nm (FIG. 2).

Separation of glycosylated and nonglycosylated C-IgG1 Fc 3. A small amount (less than 5%) of the C-IgG1 Fc 3 produced as described above was nonglycosylated, and the nonglycosylated C-IgG1 Fc was separated from the glycosylated C-IgG1 Fc using phenyl sepharose chromatography. The Protein G column purified C-IgG1 Fc was dialyzed against Buffer A (20 mM sodium phosphate, 1 M ammonium sulfate pH 7.0) at 4° C. overnight. Next the protein was loaded onto a phenyl sepharose column (50 mL bed volume, pre-equilibrated with Buffer A), and a 600 mL linear gradient starting with Buffer A and ending with Buffer B (20 mM sodium phosphate, 0 M ammonium sulfate pH 7.0) was then used to elute the C-IgG1 Fc protein. Fractions (5 mL) were collected throughout the gradient and analyzed by SDS-PAGE gel. The fractions containing only glycosylated C-IgG1 Fc were pooled and dialyzed against 20 mM sodium phosphate pH 7.0 at 4° C. overnight resulting in fully glycosylated C-IgG1 Fc 3 (FIG. 2).

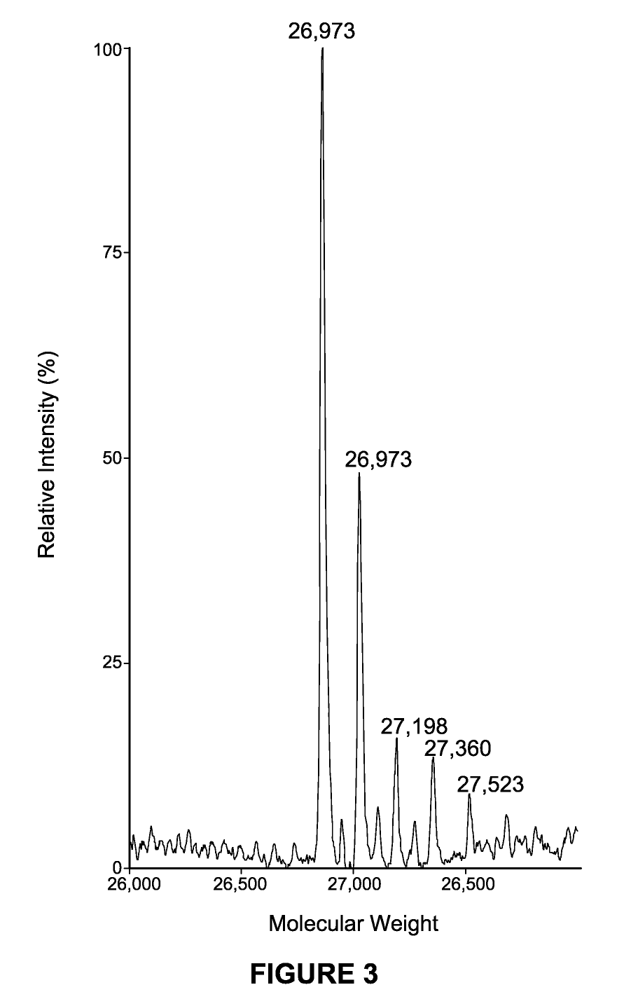

Characterization of heterogeneously glycosylated C-IgG1 Fc 3. The resulting heterogeneously glycosylated C-IgG1 Fc 3 was analyzed by electrospray mass spectrometry after reducing the disulfide bonds. A 1 mg sample of heterogeneously glycosylated C-IgG1 Fc 3 was reduced with 5 mM DTT, and then DTT and salt in the sample were removed using C18 reverse phase HPLC (10-90% acetonitrile in H₂O, 0.1% TFA). The resulting reduced, desalted C-IgG1 Fc glycoprotein (3) was then analyzed by ESI mass spectroscopy (FIG. 3). The ESI mass spectra indicated the presence of high mannose C-IgG1 Fc glycoforms containing 8-12 mannose residues (Table 1).

TABLE 1
Calculated and observed masses of C-IgG1 Fc
glycoforms from ESI MS.

| | Calculated | Observed |
|---|---|---|
| C-IgG1 Fc glycoform | mass | mass |
| Deglycosylated C-IgG1 Fc | 25,169 | 26,168 |
| Man₈GlcNAc₂-C-IgG1 Fc | 26,872 | 26,873 |
| Man₉GlcNAc₂-C-IgG1 Fc | 27,035 | 27,034 |
| Man₁₀GlcNAc₂-C-IgG1 Fc | 27,198 | 27,198 |
| Man₁₁GlcNAc₂-C-IgG1 Fc | 27,361 | 27,360 |
| Man₁₂GlcNAc₂-C-IgG1 Fc | 27,524 | 27,523 |

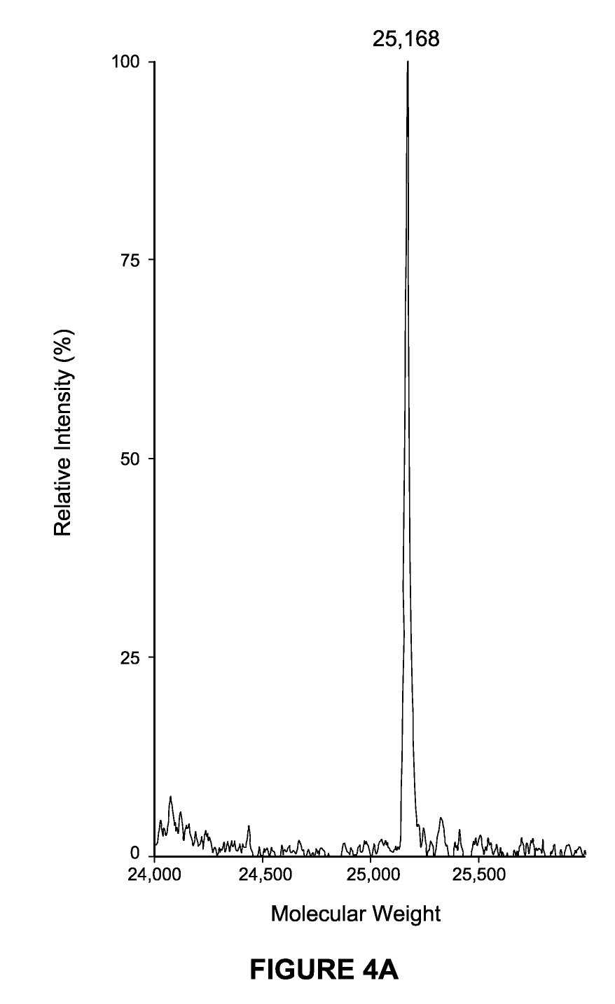

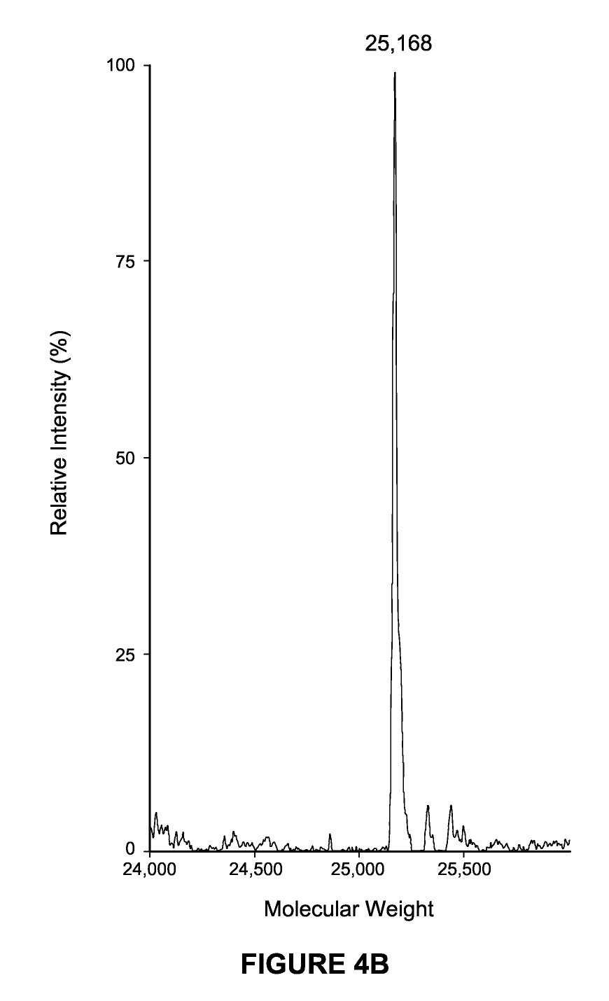

Deglycosylation of C-IgG1 Fc 3. To confirm that the heterogeneity of glycosylated C-IgG1 Fc 3 was due to the attached heterogeneous N-linked oligosaccharide, glycosylated C-IgG1 Fc was deglycosylated with PNGase F and then analyzed by ESI MS. 100 units of PNGase F (5 μl) were added into 1 mL of 2 mg/mL of glycosylated C-IgG1 Fc 3. After 12 h incubation at room temperature, the reaction was dialyzed against 20 mM sodium phosphate, 200 mM hydroxylamine pH 5.0 at room temperature for 8 h, and then dialyzed against 20 mM sodium phosphate pH 7.0 at 4° C. overnight. The hydroxylamine treated, deglycosylated C-IgG1 Fc was then reduced with 5 mM DTT, desalted by C18 reverse phase HPLC (10-90% acetonitrile in H₂O, 0.1% TFA), and then analyzed by ESI mass spectroscopy (FIG. 4A). Removal of the N-linked oligosaccharide by PNGase F greatly simplifies the mass spectra resulting in only a single peak for the deglycosylated C-IgG1 Fc protein (calcd 25169, found 26168). This procedure was also carried out without the hydroxylamine treatment step to give deglycosylated C-IgG1 Fc without hydroxylamine treatment (FIG. 4B)

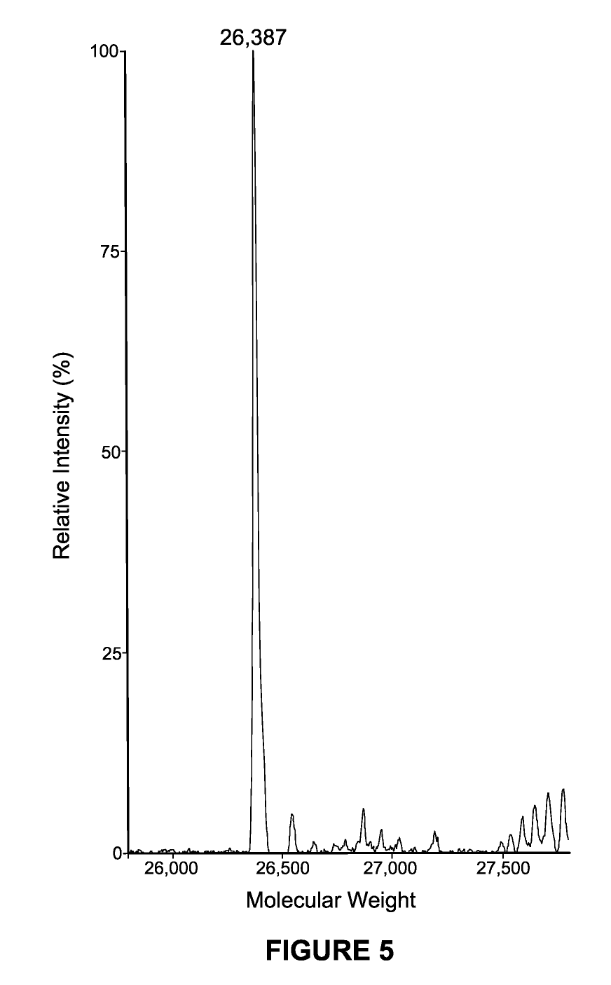

α-Mannosidase IA digestion of C-IgG1 Fc 3 to produce C-Man₅-IgG1 Fc 4. 2 mL of 2 mg/mL of glycosylated C-IgG1 Fc 3 was placed into a 6000 MWCO dialysis bag and dialyzed against 10 mM MES, 5 mM CaCl₂, 150 mM NaCl pH 6.6 at 4° C. overnight. α-Mannosidase IA (500 units, 50 μL) was added into the dialysis bag and dialyzed against 10 mM MES, 5 mM CaCl₂, 150 mM NaCl pH 6.6 at room temperature for 24 h. After 24 h digestion, the protein was dialyzed against 20 mM sodium phosphate, 200 mM hydroxylamine pH 5.0 at room temperature for 8 h, and then dialyzed against 20 mM sodium phosphate pH 7.0 at 4° C. overnight. A sample (500 μL) of the α-mannosidase IA and hydroxylamine treated C-IgG1 Fc was reduced with 5 mM DTT, desalted by C18 reverse phase HPLC (10-90% acetonitrile in H₂O, 0.1% TFA), and then analyzed by ESI mass spectroscopy (FIG. 5). The resulting mass spectra showed conversion of the heterogeneously glycosylated C-IgG1 Fc 3 into a single glycoform containing 5 mannose residues, hereafter referred to as C-Man₅-IgG1 Fc 4 (calcd 26386, found 26387).

### 3. Synthesis and Characterization of Cyclic-RGDfK 1 and Cyclic-RGDfK-Thioester 2

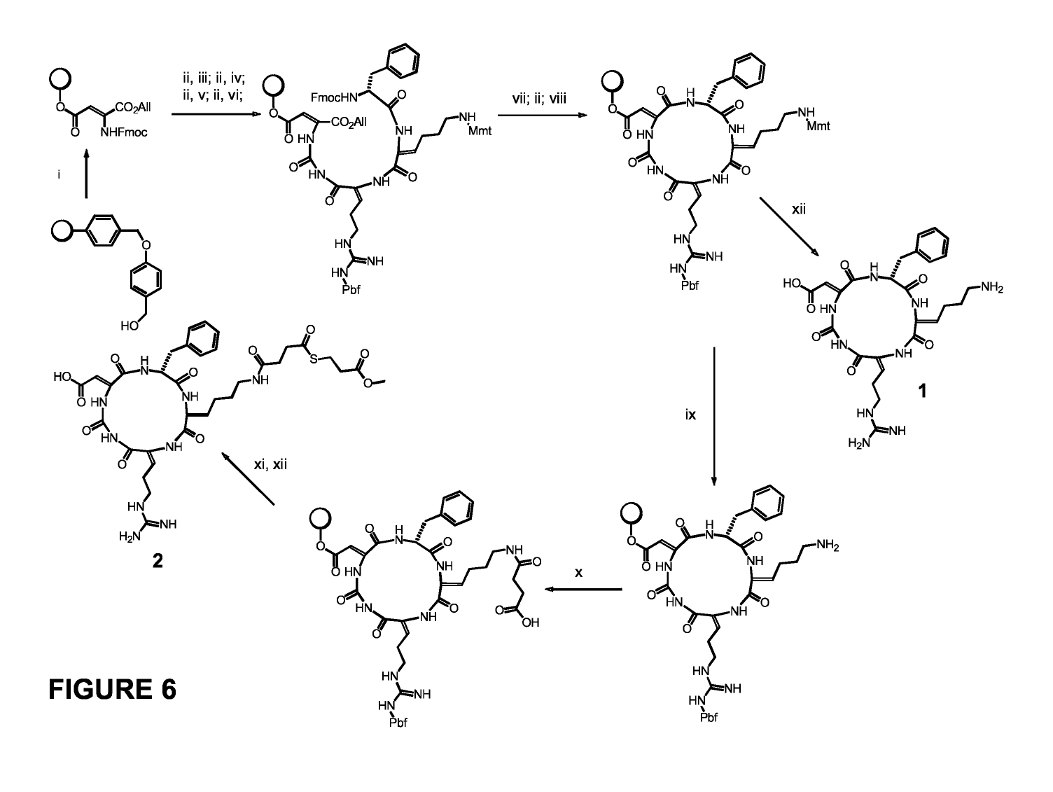

Solid phase synthesis of cyclic-RGDfK 1 (SEQ ID 3). (This synthesis is summarized in FIG. 6.) Wang resin (100-200 mesh, 0.90 mmole/g) was used and the side chain of Aspartic acid was loaded on the resin by using 3 equivalents of Fmoc-Asp-OAII, 3 equivalents of DIC, 3 equivalents of HOBt and 1.1 equivalents of DMAP in DMF (i, ii, FIG. 6). Next, Gly, Arg(pbf), Lys(mmt) and D-Phe were manually coupled in sequential order following the standard Fmoc peptide synthesis procedures using 5 equivalents of Fmoc-amino acids, 5 equivalents of DEPBT, and 2 equivalents of DIEA in DMF (iii, iv, v, and vi, FIG. 6). The C-terminal allyl ester protecting group was then removed by treatment with Pd(PPh₃)₄ in CHCl₃/AcOH/N-methylmorpholine 37:2:1 for 3 h under a nitrogen atmosphere (vii, FIG. 6). The resin was washed carefully with DIPEA (5%) in DMF, and then the N-terminal Fmoc protecting group was removed by treatment with 20% piperidine in DMF (Wellings, D. A. et al., Methods Enzymol. 1997, 289, 44-67). 5 equivalents of DEPBT and 2 equivalents of DIEA were then used to cyclize the peptide on resin, and the cyclization reaction was carried out for 18 hours in DMF (viii, FIG. 6). A ninhydrin test was used to determine the completion of the cyclization reaction (Kaiser, E. et al., Anal. Biochem. 1970, 34, 595-598). The cyclic peptide was cleaved from resin by treatment with neat TFA with TIS as scavenger (xii, FIG. 6) and purified by RP-HPLC (Agilent Zorbax SB-CB C8, prep., 21.2×259 mm) to give 0.45 mg pure 1. Yield 6.2%, according to the loading of the resin reported by the manufacturer. tR=1.383 min, ESI-MS: m/z=604.5 calculated mass for C27H42N9O7⁺[M+H]⁺=604.7.

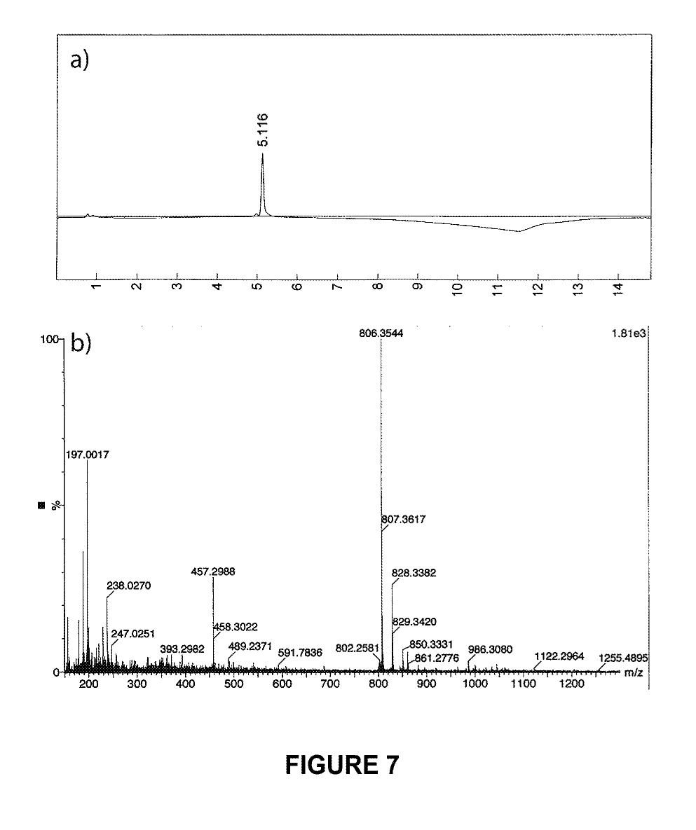

Solid phase synthesis of cyclic-RGDfK thioester 2 (SEQ ID 4). To produce the cyclic-RGDfK thioester 2 (SEQ ID 4), Wang resin with cyclic-RGDfK was prepared as described above, omitting cleavage from the resin and purification steps (FIG. 6). The resin with the cyclic-RGDfK (SEQ ID 4) was then treated with 1% TFA in DCM to remove the Mmt protecting group on the side chain, and then the resin was washed thoroughly with DCM (ix, FIG. 6). Then, 10 mole equivalents of succinate anhydride and 3 equivalents of N-methylmorpholine were added to the peptide synthesis vial and the reaction was carried out for 4 hours with shaking (x, FIG. 6). A ninhydrin test was used to determine the completion of the reaction. Next 10 equivalents 3-mercaptopropanoate were used to produce the corresponding cyclic-RGDfK thioester 2 using 5 equivalents of HOBt, 5 equivalents of DIC, and 1 equivalent of DMAP (xi, FIG. 6). The thioester formation reaction was carried out 2 times, 12 hours each time, under nitrogen. The peptide thioester was then cleaved from the resin by treatment with neat TFA with TIS scavenger (xii, FIG. 6). The crude peptide thioester 2 was precipitated and washed with diethyl ether and purified by RP-HPLC (Agilent Zorbax SB-CB C8, prep., 21.2×259 mm) to give 2.80 mg of the pure thioester 2, 6.0% yield according to the loading of the resin reported by the manufacturer. This synthesis is summarized in FIG. 6. tR=5.116 min analytical HPLC, (FIG. 7), (10-80% B in 10 min, Beckman SGB 0.46×5 cm Zorbax C8 column, A buffer Water, 0.1% TFA; B buffer, Acetonitrile, 0.1% TFA). ESI-MS m/z=806.5, calculated mass for C35H52N9O11S⁺, [M+H]⁺=806.9; HRMS ESI-TOF [M+H]⁺ calcd. 806.3507, found 806.3544.

### 4. NCL to Produce RGD-Man₅-IgG1 Fc 5

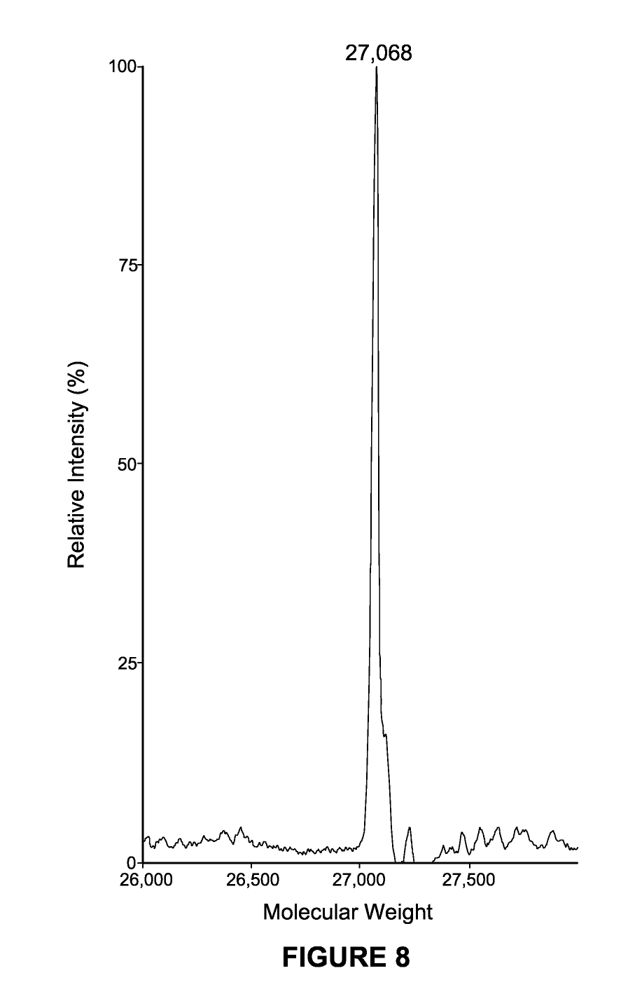

NCL of cyclic-RGDfK-thioester 2 to C-Man₅-IgG1 Fc 4 to produce 5. 1.8 mL of 2 mg/mL of α-mannosidase 1A and hydroxylamine treated C-Man₅-IgG1 Fc 4 (prepared as described above) was dialyzed against ligation buffer (5 mM betaine, 20 mM sodium phosphate pH 7.5) at 4° C. for 12 h, and then transferred into an eppendorf tube for native chemical ligation. The ligation reaction was initiated by adding 4 mg of cyclic-RGDfK-thioester 2 (SEQ ID 4) (2.6 mM final concentration) and 200 μL of 300 mM sodium 2-mercaptoethanesulfonate (30 mM final concentration) into the C-Man₅-IgG1 Fc 4 solution, and this mixture was incubated at room temperature for 24 h. After 24 h, the mixture was dialyzed against 20 mM sodium phosphate pH 7.5 at 4° C. for 12 h to remove the excess thioester. 500 μL of the ligation product was reduced with 5 mM DTT and desalted by C18 reverse phase HPLC (10-90% acetonitrile in H₂O, 0.1% TFA) and then analyzed by ESI mass spectroscopy (FIG. 8; cRGD-Man₅-C-IgG Fc 5, calcd. 27071, found 27070).

### 5. Bioassays

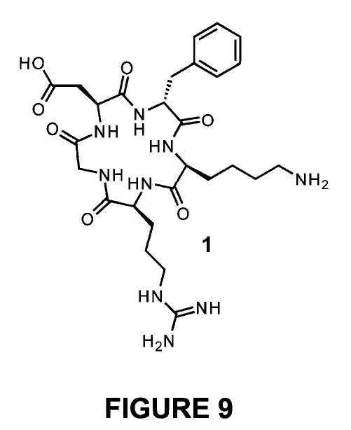

Integrin adhesion assay. A Corning Costar EIA/RIA 96-well plate was coated with the following protein solutions (100 μl/well): 10 mg/mL bovine serum albumin (BSA), 5 μg/mL human fibrinogen (a natural ligand of the αᵥβ₃ integrin receptor), 5 μg/mL C-IgG1 Fc, and 5 μg/mL RGD-IgG1 Fc all in phosphate buffered saline (PBS: 137 mM NaCl, 2.7 mM KCl, 10 mM sodium phosphate dibasic, 2 mM potassium phosphate monobasic, pH=7.4). After incubation overnight at 4° C., the protein solutions were removed from the wells and all of the wells were blocked with 150 μl of 10 mg/mL BSA in PBS for 1 h. at 37° C. The BSA was removed from the wells and each well was washed with 3×200 μl PBS buffer. The plate was tapped dry and WM-115 melanoma cells prepared according to the method of Wu et al. (Methods in Molecular Biology, 1999, 129, 211-217) were added to each of the wells at approximately 30,000 cells (3×10⁵ cells/mL) in 100 μl of assay medium (assay medium: E-MEM, 20 mM HEPES at pH 7.5, 1.5 mM MgCl₂, 1 mM CaCl₂, 1.5 mM MnCl₂, 0.1% BSA). The plate was incubated at 37° C. for 30 minutes and then wells were washed with 3×200 μl PBS buffer to remove non-adhering cells. The cells remaining in the wells after washing were fixed with 70% aqueous ethanol for 30 minutes, and then stained with 0.2% crystal violet in 0.75 M borate buffer pH at 6.0 for 30 minutes. Excess crystal violet was washed away with distilled water (3×200 μl), and then crystal violet from stained cells was solubilized with 100 μl of 1% SDS. The amount of crystal violet in each well was quantified at 540 nm with Titertek Multiscan plate reader. The results from this experiment are summarized in FIG. 9A.

Integrin adhesion inhibition assay. To further investigate the ability of the RGD linked IgG1 Fc proteins to antagonize the αᵥβ₃ integrin receptor, adhesion inhibition assays were conducted comparing the cyclic-RGDfK peptide 1 (SEQ ID 3) to the IgG1 Fc linked cyclic-RGD peptide 5. Utilizing the method of Wu et al., 96-well plates were coated with 5 μg/mL fibrinogen and blocked with BSA as described in the integrin adhesion assay above. WM-115 melanoma cells prepared according to the method of Wu et al. were added to wells in the presence of serial dilutions of either the free cyclic-RGD integrin antagonist 1 or the protein bound RGD-IgG1 Fc 5. Incubation, washing, cell fixing, and crystal violet staining were conducted as described above. Fitting adhesion inhibition data for cyclic peptide 1 and antibody fragment 5 to a one-site competitive pharmacology model as described by Wu et al. yielded IC₅₀'s for inhibition of cellular adhesion of 102 nM for the free cyclic-RGD integrin antagonist 1 and 104 nM for the protein bound RGD-IgG1 Fc 5.

### 6. Fluorescence Microscopy

Protein labeling with FITC. Proteins, C-IgG1 Fc 3 or RGD modified C-IgG1 Fc 5 (800 μL of 2 mg/mL), were dialyzed against 20 mM sodium phosphate pH 7.4 at 4° C. for 12 h and then transferred into separate eppendorf tubes. The labeling reaction was initiated by adding 8 μL of a 10 mg/mL solution of FITC in DMSO, and the reaction was incubated at room temperature for 24 h. After the reaction, the FITC labeled proteins, C-IgG1 Fc 3 or cRGD modified C-IgG1 Fc 5, were dialyzed against 20 mM tris-base buffer pH 7.0 at 4° C. for 24 h to remove the excess FITC. The degree of protein labeling (FITC:protein molar ratio) was determined by the absorbance at 280 nm and 495 nm using the following expressions as recommended by Thermo Scientific-Pierce Protein Research Products:

Protein concentration (M)=(A₂₈₀−A₄₉₅×CF)/εₚᵣₒₜₑᵢₙ εₚᵣₒₜₑᵢₙ=protein molar extinction coefficient (35791 M⁻¹ cm⁻¹ for C-IgG Fc) CF=correction factor (0.3 for FITC)

Degree of protein labeling=A₄₉₅/(ε_{fluor}×protein concentration)ε_{fluor}=70000 M⁻¹ cm⁻¹ for FITC

The degrees of protein labeling with FITC were 2.8 and 2.2 for C-IgG1 Fc 3 (FITC-3) and cRGD modified C-IgG1 Fc 5 (FITC-5) respectively.

Fluorescent microscopy. WM-115 cells were seeded into a 6-well cell culture plate (4×10⁵ cells/well) containing three 12 mm poly-L-lysine coated coverslips in each well. The cells were grown on the coverslips in an incubator at 37° C. with 5% CO₂ for 12 h, and then the media was aspirated and the cells were washed with PBS twice. MEM media (1.9 mL) was added into all of the wells, and then 0.1 mL of 20 μM FITC labeled C-IgG1 Fc 3 was added into three wells and 0.1 mL of 20 μM FITC labeled RGD-IgG1 Fc 5 was added into the other three wells. After a 15 min incubation at 37° C., the media was aspirated and the cells were washed with PBS three times. After washing with PBS, the coverslips were fixed in 4% paraformaldehyde for 10 min and then washed with PBS twice before being mounted in aqueous mounting medium with anti-fading agents. All images were acquired on a Nikon Eclipse 90i, 40× Plan Apo objective (NA 1.3) and a CoolSnap HQ CCD camera (Photometrics, Tucson, Ariz.). The camera and filters were controlled by Metamorph (Molecular Devices, Sunnyvale, Calif.). Images were processed in Adobe Photoshop CS for control and experimental samples. The background subtracted fluorescence intensities were determined from three independent experiments. In each experiment, roughly 50 cells were measured for each condition (FITC-3 or FITC-5) and fluorescence intensities of cells were determined using Metamorph. Average fluorescent intensity with background subtracted from the three independent experiments described above are given in Table 2. The average fluorescence intensities of cells incubated with FITC labeled RGD-IgG1 Fc 5 was significantly greater (approximately 5 times larger) than cells incubated with FITC labeled C-IgG1 Fc 3.

TABLE S2
Average Fluorescence intensities of WM-115 cells
incubated with FITC labeled 3 or FITC labeled 5.

| | Average Fluorescence | Standard |
|---|---|---|
| | intensity | Deviation |
| FITC-C-IgG1 Fc 3 | 21.7 | 3.3 |
| FITC-RGD-IgG1 Fc 5 | 112.9 | 13.2 |

### Results

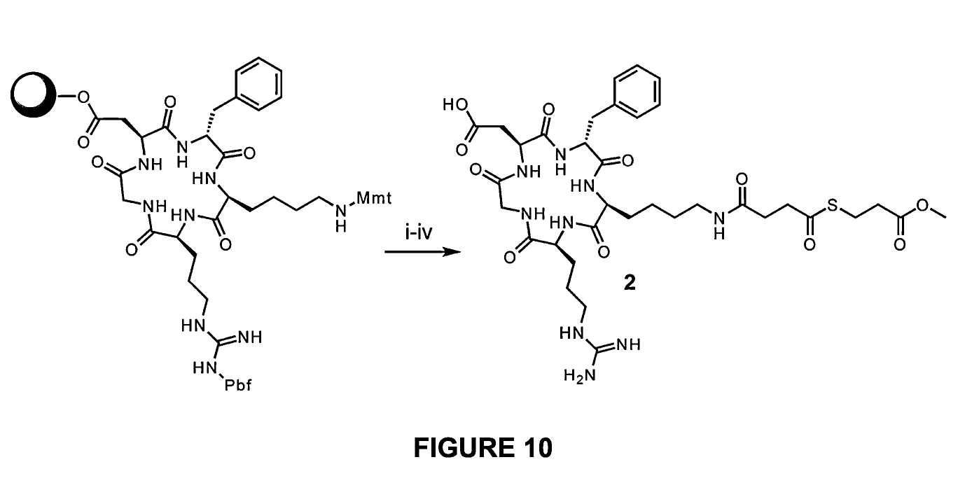

The strategy for targeting antibody Fc regions to bind to cancer cells was to link ligands of cell surface receptors highly expressed on cancer cells to the N-terminus of IgG1 Fc. In this way the ligands were displayed on the IgG1 Fc in a manner similar to how the Fabs would be displayed on a full-length antibody. Ligand binding to cellular receptors should then mimic antibody binding to antigen, displaying the Fc region in a way that should allow antibody effector functions such as antibody dependent cell-mediated cytotoxicity (ADCC) and complement dependent cytotoxicity (CDC) to function. For a receptor ligand to attach to IgG1 Fc the cyclic RGD peptide 1 was chosen (FIG. 9), a selective antagonist of the αᵥβ₃ integrin receptor. The αᵥβ₃ integrin receptor is overexpressed in many types of cancer and also involved in angiogenesis making it a good target for delivering therapeutics to cancer cells and for imaging. To achieve attachment of receptor ligands such as 1 site-selectively on the N-terminus of IgG1 Fc NCL was chosen because it is a chemoselective reaction that will specifically modify the N-terminus of proteins containing N-terminal cysteines, linking proteins to synthetic molecules with a stable peptide bond without forming additional stereocenters. To use NCL for this application it was necessary to develop methods for expressing the IgG1 Fc glycoprotein with a N-terminal cysteine and for producing cyclic RGD thioester derivative 2 (FIG. 10). In addition, to overcome the natural heterogeneity of glycoproteins produced in most cell lines, a glycosylation deficient yeast strain and enzymatic digestion to produce a homogeneously glycosylated IgG1 Fc glycoprotein was utilized, which greatly simplified the monitoring of glycoprotein modification reactions with mass spectrometry.

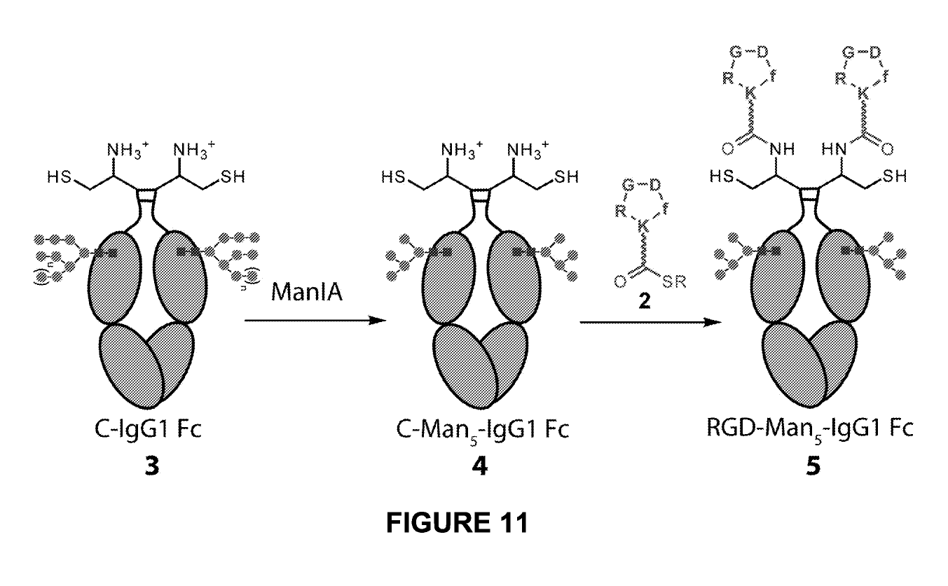

NCL is a chemical reaction between N-terminal cysteines and thioesters which forms native peptide bonds (Dawson, P. E. et al., Science 1994, 266, 776-779). Methods for expression of glycoproteins containing N-terminal cysteines have not been reported, so a method to express the IgG1 Fc glycoprotein with an N-terminal cysteine was worked out. As an N-linked glycoprotein, IgG1 Fc must pass through the secretory pathway in order to be glycosylated, which necessitates the presence of an N-terminal signal peptide in the protein sequence to direct IgG1 Fc into the secretory pathway. In addition, for efficient secretion of proteins from yeast cells, an N-terminal secretion signal, the α-factor, is required. As a result an N-terminal cysteine containing IgG1 Fc cannot be directly expressed, but must also contain these additional peptide sequences. Since the N-terminal signal peptide and α-factor secretion signal are proteolytically removed during protein expression, the possibility of using the endogenous yeast protease involved in the last step of this process, the Kex2 protease, was explored to generate an N-terminal cysteine form of the IgG1 Fc glycoprotein. IgG1 Fc was expressed with a cysteine residue immediately following the Kex2 protease cleavage site (FIG. 1). This approach worked well and expression of this construct in Pichia pastoris resulted in production of N-terminal cysteine containing IgG1 Fc, referred to as C-IgG1 Fc 3 (FIG. 11), as characterized by electrospray mass spectrometry (ESI MS) after PNGase F treatment to remove the heterogeneous N-linked oligosaccharide (FIGS. 4A and B). One concern with this approach is that generation of reactive N-terminal cysteines during yeast secretory expression has the potential of forming thiazolidine adducts through the reaction of the N-terminal cysteines with aldehyde or ketone containing metabolites. Little thiazolidine formation was observed by mass spectrometry of the PNGase F treated C-IgG1 Fc, but to insure free N-terminal cysteines for NCL reactions the glycoproteins used in this research were treated with hydroxylamine to hydrolyze potential thiazolidines prior to NCL reactions (FIGS. 4A and B). To produce homogeneously glycosylated, N-terminal cysteine containing IgG1 Fc, the C-IgG1 Fc glycoprotein 3 was expressed in a glycosylation deficient yeast strain produced in our laboratory using the methods of Nett and Gerngross (Yeast 2003, 20, 1279-1290). Expression in the glycosylation deficient yeast strain resulted in a mixture of high mannose C-IgG1 Fc glycoforms containing between 8 to 12 mannose residues (FIG. 3). Treatment of the heterogeneously glycosylated 3 with α-1,2-mannosidase IA (ManIA) to remove α-1,2 linked mannose residues produced a homogeneous glycoform of C-IgG1 Fc containing 5 mannose residues (C-Man₅-IgG1 Fc 4), confirmed by mass spectrometry (FIG. 5). This homogeneous C-Man₅-IgG1 Fc 4 was then used in NCL reactions.

The synthesis of the cyclic RGD thioester integrin antagonist 2 was based upon the solid phase synthesis of cyclic RGD derivatives reported by McCusker et al. (Bioorg. Med. Chem. Lett. 2002, 547-549), except that a monomethoxytrityl (Mmt) side chain protected lysine was utilized (FIG. 10). After on-resin construction of the cyclic-(RGDfK) peptide (SEQ ID 4), the Mmt protecting group of the lysine was selectively deprotected with 1% TFA in CH₂Cl₂, and then the lysine amine was acylated with succinic anhydride. Thioester formation was effected on-resin by addition of methyl 3-mercaptopropionate, DMAP, and HOBt using DIC as a coupling reagent. Cleavage from the resin and removal of the remaining protecting groups was accomplished using TFA with triisopropylsilane (TIS) as a scavenger. The resulting peptide thioester 2 was precipitated from diethylether and purified by reverse phase HPLC.

Ligation of C-Man₅-IgG1 Fc 4 to thioester 2 was carried out by mixing C-Man₅-IgG1 Fc 4 (2 mg/mL, 76 μM) with 2.6 mM thioester 2 in 20 mM sodium phosphate buffer pH 7.5 containing 5 mM betaine and 30 mM sodium 2-mercaptoethanesulfonic acid (MESNA). The reaction was incubated for 24 hours and then an aliquot of the ligation reaction was analyzed by ESI MS. As can be seen in FIGS. 3, 5 and 11 the ligation proceeded well and was greater than 95% complete after 24 hours as estimated by ESI MS.

To determine if RGD-Man₅-IgG1 Fc 5 was targeted to bind αᵥβ₃ integrin receptor expressing cells, experiments were conducted using WM-115 melanoma cells which express the αᵥβ₃ integrin receptor at a high level (Wu, J. M. et al., Methods Mol. Biol. 1999, 129, 211-217). First, an adhesion assay was conducted taking advantage of the fact that antibodies and antibody fragments such as IgG1 Fc will coat the hydrophobic surfaces of ELISA plates allowing ELISA type binding assays to be conducted. The ability of RGD-Man₅-IgG1 Fc 5 coating of ELISA microplate wells to promote adhesion of WM-115 cells was compared to wells coated with fibrinogen (a natural ligand for the αᵥβ₃ integrin receptor), C-IgG1 Fc 3, and bovine serum albumin (BSA). Wells containing C-IgG1 Fc 3 alone had few cells adhering to them and were similar to the negative control BSA wells. Interestingly, the wells coated with RGD-Man₅-IgG1 Fc 5 had the highest amounts of cells adhering to them, approximately 14-fold more than IgG1 Fc alone and also more than 2 times more than adhered to the natural ligand fibrinogen coated wells. This indicates strong binding interactions between RGD-Man₅-IgG1 Fc 5 coating the wells and the αᵥβ₃ integrin receptors on WM-115 cells.

To further assess the ability of the RGD modified IgG1 Fc 5 to bind to the αᵥβ₃ integrin receptor, adhesion inhibition assays were conducted comparing 5 to the free RGD peptide antagonist 1 using the methods of Wu et al. (ibid). Interestingly, the RGD modified IgG1 Fc 5 had a nearly identical IC₅₀ to the free cyclic RGD peptide 1, 104 nM and 102 nM respectively. This indicates that the cyclic RGD modification of IgG1 Fc is capable of targeting the antibody fragment to bind to αᵥβ₃ integrin receptor expressing cancer cells in a manner similar to the free peptide.

Fluorescence microscopy experiments were also conducted to observe the binding of the RGD modified IgG1 Fc to WM-115 cells. Both C-IgG1 Fc 3 and RGD-IgG1 Fc 5 were labeled with fluorescein isothiocyanate (FITC), and utilized in this experiment. WM-115 cells grown on glass cover slips were incubated with 1 μM of either FITC labeled C-IgG1 Fc 3 or FITC labeled RGD-IgG1 Fc 5 for 15 minutes at 37° C. Cells were then washed, fixed, cell nuclei were stained and cells were mounted for fluorescence microscopy. There was a significant difference between the binding of FITC labeled RGD-IgG1 Fc 5 when compared to FITC labeled C-IgG1 Fc 3, confirming the RGD ligand is active for binding to the αᵥβ₃ integrin receptor. These results have been quantified (Table 2) and are similar to what has been observed for RGD-dendrimers binding to the αᵥβ₃ integrin receptor, and may indicate endocytosis of the RGD-IgG1 Fc 5 (Boturyn, D. et al., J. Am. Chem. Soc. 2004, 126, 5730-5739).

In summary, methods to N-terminally modify glycosylated IgG1 Fc produced in yeast have been developed and applied to the N-terminal attachment of a bioactive receptor ligand, a cyclic RGD αᵥβ₃ integrin receptor antagonist. Homogenously glycosylated C-Man₅-IgG1 Fc 4 with a N-terminal cysteine was produced by expression in a glycosylation deficient yeast strain, utilizing Kex2 proteolytic processing to generate the N-terminal cysteine on the glycoprotein in the secretory pathway, and in vitro mannosidase IA digestion to produce the homogeneous Man₅ glycoform. A thioester containing cyclic RGD integrin antagonist 2 was produced by solid phase peptide synthesis and attached to the N-terminus of C-Man₅-IgG1 Fc 4 utilizing NCL. The resulting RGD-Man₅-IgG1 Fc glycoprotein 5 retained its ability to bind to and antagonize the αᵥβ₃ integrin receptor on WM-115 melanoma cells as demonstrated by adhesion assay, adhesion inhibition assay, and fluorescence microscopy. Antibody fragments such as IgG1 Fc have significant bioactivity in their own right, with long circulatory half-lives and the ability to direct antibody dependent immune responses such as ADCC and CDC, and so attachment of cell receptor ligands to IgG1 Fc in this manner may augment the biological activity of the attached ligands and this possibility is under further investigation.

The techniques developed here for NCL modification of expressed glycoproteins are general and may be applied to the modification of other glycoproteins or for the production of glycosylated protein fragments for the chemoenzymatic synthesis of homogeneously glycosylated glycoproteins.

It should be understood, of course, that the foregoing relates to exemplary embodiments of the invention and that modifications may be made without departing from the spirit and scope of the invention as set forth in the following claims.

We claim:

1. A method for expression of a glycoprotein comprising a N-terminal cysteine, the method comprising the steps of:
inserting a nucleotide sequence encoding for the glycoprotein into a plasmid to form a plasmid construct, wherein the nucleotide sequence comprises a codon of cysteine at the N-terminus and wherein the plasmid construct further comprises nucleotide sequences encoding a N-terminal signal peptide and a secretion signal;
transforming the plasma construct into a glycosylation deficient strain of yeast;
culturing the yeast transformed with the plasma construct in culture media to express the glycoprotein.

2. The method of claim 1 further comprising the steps of:
isolating the glycoprotein from the culture media; and
cleaving the N-terminal signal peptide and secretion signal from the glycoprotein.

3. The method of claim 2 wherein the N-terminal signal peptide and secretion signal are cleaved from the glycoprotein using a protease.

4. The method of claim 1 wherein the plasmid comprises the N-terminal signal peptide or the secretion signal.

5. The method of claim 1 wherein the secretion signal is α-factor

6. The method of claim 1 wherein the glycoprotein is a Fc antibody fragment.

7. The method of claim 6 wherein the Fc antibody fragment is IgG1 Fc glycoprotein.

8. The method of claim 1 wherein the glycosylation deficient strain of yeast is an och1 knockout mutant of Pichia pastoris.

9. A glycoprotein comprising a N-terminal cysteine made by the method of claim 1.

10. A method of making a chemically modified glycoprotein fragment comprising the steps of:
providing a glycoprotein comprising a N-terminal cysteine; and
reacting the glycoprotein with a ligand, wherein the ligand comprises a thioester.

11. The method of claim 10 wherein the glycoprotein is a Fc antibody fragment.

12. The method of claim 11 wherein the Fc antibody fragment is IgG1 Fc glycoprotein.

13. The method of claim 10 wherein the ligand is a cyclic RGD peptide thioester.

14. The method of claim 10 wherein the chemically modified glycoprotein fragment is made by the method of:
inserting a nucleotide sequence encoding for the glycoprotein into a plasmid to form a plasmid construct, wherein the nucleotide sequence comprises a codon of cysteine at the N-terminus and wherein the plasmid construct further comprises nucleotide sequences encoding a N-terminal signal peptide and a secretion signal;
transforming the plasma construct into a glycosylation deficient strain of yeast;
culturing the yeast transformed with the plasma construct in culture media to express the glycoprotein.

15. A chemically modified glycoprotein fragment made by the method of claim 10.

16. The chemically modified glycoprotein fragment of claim 15 wherein the glycoprotein fragment is IgG1 Fc and the ligand is a cyclic RGD peptide thioester.

17. A method of killing cancer cells comprising the step of contacting the cancer cells with an effective amount of the chemically modified glycoprotein fragment of claim 16.

18. A method of treating a patient having cancer comprising the step of administering to the patient a therapeutically effective amount of the chemically modified glycoprotein fragment of claim 16.


SEQUENCE LISTING (4 sequences)

SEQ ID NO: 1 - DNA, 42 bp, Homo sapiens
ggcccgctcgagaaaagatgcacatgcccaccgtgcccagca

SEQ ID NO: 2 - DNA, 41 bp, Homo sapiens
gggcccgcggcggccgctcatttacccggagacagggagag

SEQ ID NO: 3 - protein, 5 aa, Artificial Sequence
  Based on known RGD binding sequence
RGDFK

SEQ ID NO: 4 - protein, 5 aa, Artificial Sequence
  Based on known RGD binding peptide
RGDFK